(19) United States
(12) Patent Application Publication
(10) Pub. No.: US 2025/0186604 A1
(43) Pub. Date: Jun. 12, 2025

(54) ANTI-PSMA ANTIBODY CONJUGATES

(71) Applicant: ADC THERAPEUTICS SA, Epalinges (CH)

(72) Inventors: Paul HOGG, London (GB); Nicolas VEILLARD, London (GB); Patricius Hendrikus Cornelis VAN BERKEL, London (GB)

(21) Appl. No.: 18/969,940

(22) Filed: Dec. 5, 2024

(30) Foreign Application Priority Data
Dec. 6, 2023 (EP) 23214756.1
Apr. 4, 2024 (EP) 24168598.1

(51) Int. Cl. A61K 47/68; A61P 35/00; C07K 16/30

(52) U.S. Cl. CPC A61K 47/68037; A61K 47/6869; A61K 47/6889; A61P 35/00; C07K 16/3069

(57) ABSTRACT

This disclosure relates to antibody conjugates comprising an antibody that binds specifically to the PSMA protein, conjugated to an Exatecan, and associated therapeutic uses.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 17KB file named “0233-0035US1_SL.xml,” created on Dec. 1, 2024.

### FIELD OF THE INVENTION

The present disclosure relates to anti-PSMA drug conjugates and their use in therapy.

### BACKGROUND

PSMA is present on the cell surface of some normal prostatic epithelial cells, normal renal proximal tubular cells, proximal small bowel and some astrocytes (found in the brain). PSMA is highly upregulated/overexpressed on prostate cancer (Pea) cells. Expression levels of PSMA increase along with prostate cancer progression and PSMA levels in early stage prostate cancer predict a higher likelihood of recurrence. Furthermore, many solid tumours express PSMA in their tumour neo-vasculature whereas normal vascular endothelium is PSMA-negative.

Prostate cancer is one of the most common causes of cancer deaths in American males. In 2007, approximately 219,000 new cases are expected to be diagnosed as well as 27,000 deaths due to this disease. There is currently very limited treatment for prostate cancer once it has metastasized (spread beyond the prostate). Systemic therapy is limited to various forms of androgen (male hormone) deprivation. While most patients will demonstrate initial clinical improvement, virtually inevitably, androgen-independent cells develop. Endocrine therapy is thus palliative, not curative. Median overall survival in these patients where androgen-independent cells have developed was 28-52 months from the onset of hormonal treatment. Subsequent to developing androgen-independence, only taxane-based (i.e., docetaxel) chemotherapy has been shown to provide a survival benefit, with a median survival of 19 months. Once patients fail to respond to docetaxel, median survival is 12 months.

Where prostate cancer is localized and the patient's life expectancy is 10 years or more, radical prostatectomy offers the best chance for eradication of the disease. Historically, the drawback of this procedure is that many cancers had spread beyond the bounds of the operation by the time they were detected. However, the use of prostate-specific antigen testing has permitted early detection of prostate cancer. As a result, surgery is less extensive with fewer complications. Patients with bulky, high-grade tumours are less likely to be successfully treated by radical prostatectomy. Radiation therapy has also been widely used as an alternative to radical prostatectomy. Patients generally treated by radiation therapy are those who are older and less healthy and those with higher-grade, more clinically advanced tumours. However, after surgery or radiation therapy, if there are detectable serum prostate-specific antigen concentrations, persistent cancer is indicated. In many cases, prostate-specific antigen concentrations can be reduced by radiation treatment. However, this concentration often increases again within two years.

It has been recently shown that small molecule PSMA enzyme inhibitors could slow the growth rate of PSMA-expressing Pea cells in vitro. However, not only have these inhibitors in the past failed to have any meaningful effect on tumour cell growth in animal models, but since they act on PSMA folate hydrolase activity they have had a negative impact on whole body folate metabolism which is critical for normal physiological processes.

Accordingly, there is a need for an effective non-surgical approach to the treatment of prostate cancer and other diseases.

### SUMMARY

One approach to developing treatments for prostate cancer is the use of antibody drug conjugates that target PSMA. The present disclosure is directed to an anti-PSMA antibody conjugated to an Exatecan and its use in therapy.

Accordingly in a first aspect is provided an antibody drug conjugate of formula (I):

Ab-L-Dp (I)

wherein:

Ab is an antibody that binds to PSMA, which antibody comprises (i) an immunoglobulin heavy chain variable region having a CDR1 region with the amino acid sequence shown in SEQ ID NO: 3, a CDR2 region with the amino acid sequence shown in SEQ ID NO: 4, and a CDR3 region with the amino acid sequence shown in SEQ ID NO: 5; and (ii) an immunoglobulin light chain variable region having a CDR1 region with the amino acid sequence shown in SEQ ID NO: 6, a CDR2 region with the amino acid sequence shown in SEQ ID NO: 7, and a CDR3 region with the amino acid sequence shown in SEQ ID NO: 8;

L-Dp is a drug linker conjugated to the Ab via one or more cysteine residues wherein L comprises a polyglycine moiety, Glyₙ wherein n is from 5 to 8, and D is an Exatecan, wherein the drug linker can be cleaved under cellular conditions to release an Exatecan with following formula:

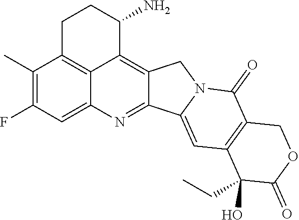 [SMILES: CC[C@@]1(O)C(=O)OCc2c1cc1n(c2=O)Cc2c-1nc1cc(F)c(C)c3c1c2[C@@H](N)CC3]

The drug loading is represented by p, the number of drug units per antibody. Drug loading may typically range from 1 to 8 Drug units (D) per antibody, such as from 1 to 6. For compositions, p represents the average drug loading of the conjugates in the composition, and p ranges from 1 to 8. In one embodiment, p ranges from 1 to 6, for example as a result of the removal/substitution of one or more cysteines, such as hinge region cysteines, in the antibody.

In a second aspect is provided the conjugate of the first aspect together with one or more pharmaceutically acceptable carriers or diluents, such as a pharmaceutical composition in liquid or lyophilized form. The composition may comprise a therapeutically effective amount of a chemotherapeutic agent.

The conjugate or compositions comprising it may be used in therapy. For example, in a method of treating a proliferative disease, the method comprising administering an effective amount of the conjugate or the composition to an individual in need of such treatment.

The treated proliferative disease may be cancer, such as prostate cancer, hepatocellular carcinoma, bladder cancer, breast cancer, colorectal cancer, gastric cancer, glioblastoma, lung cancer, lymphoma, melanoma, neuroendocrine cancer, ovarian cancer, pancreatic cancer, renal cancer, squamous cell carcinoma, or sarcoma. The proliferative disease may be characterised by the presence of a neoplasm comprising both PSMA+ve and PSMA-ve cells, and/or may be a solid tumour. The proliferative disease may be characterised by the over-expression of PSMA, either in all or most of the aberrant cells, or in at least part of the tumour neovasculature.

### DETAILED DESCRIPTION

### Antibody Drug Conjugates

In some embodiments, the present disclosure provides, inter alia, an antibody drug conjugate comprising (i) a cell binding agent which comprises the complementarity determining regions of the 2A10 antibody described herein such that the cell binding agent binds to PSMA on the surface of cell; (ii) a cytotoxin comprising Exatecan. The cytotoxin is connected to the cell binding agent via one or more linkers—examples of suitable linkers are described in more detail below. In one embodiment each linker is connected through the NH₂ on the F ring of Exatecan as described below.

Various aspects and embodiments of the disclosures herein are thus suitable for use in providing an Exatecan to a preferred site in a subject. The conjugate allows the release of an active Exatecan that does not retain any part of the linker.

Before transport or delivery into a cell, the antibody-drug conjugate (ADC) is preferably stable and remains intact, i.e. the antibody remains linked to the drug moiety. The linkers in such preferred embodiments are stable outside the target cell and may be cleaved at some efficacious rate inside the cell. An effective linker in some embodiments will: (i) maintain the specific binding properties of the antibody; (ii) allow intracellular delivery of the conjugate or drug moiety; (iii) remain stable and intact, i.e. not cleaved, until the conjugate has been delivered or transported to its targeted site; and (iv) maintain a cytotoxic, cell-killing effect or a cytostatic effect of the Exatecan drug moiety. Stability of the ADC may be measured by standard analytical techniques such as mass spectroscopy, HPLC, and the separation/analysis technique LC/MS.

### Cell Binding Agents/Antibodies

The cell binding agent is an antibody that binds to PSMA and which comprises the complementarity determining regions (CDRs) of monoclonal antibody 2A10.

The term “antibody” herein is used in the broadest sense and specifically covers monoclonal antibodies, including both intact antibodies and antibody fragments, so long as they exhibit the desired biological activity, for example, the ability to bind PSMA. Antibodies may be murine, rat, human, humanized, chimeric, or derived from other species. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2m1, A2m2, Km1, Km2 and Km3) of immunoglobulin molecule.

A variety of immunoglobulin variant formats are known in the art which are derived from conventional immunoglobulins, such as bispecific antibodies, scFvs, nanobodies and the like. These are all within the scope of the term “antibody” provided they retain the 2A10 CDRs and/or PSMA binding activity. Thus in some embodiments the antibody comprises (i) an immunoglobulin heavy chain variable region having a CDR1 region with the amino acid sequence shown in SEQ ID NO: 3, a CDR2 region with the amino acid sequence shown in SEQ ID NO: 4, and a CDR3 region with the amino acid sequence shown in SEQ ID NO: 5; and (ii) an immunoglobulin light chain variable region having a CDR1 region with the amino acid sequence shown in SEQ ID NO: 6, a CDR2 region with the amino acid sequence shown in SEQ ID NO: 7, and a CDR3 region with the amino acid sequence shown in SEQ ID NO: 8.

The CDR sequences as disclosed herein have been identified and defined using the Kabat numbering scheme (Kabat et al., U.S. Department of Health and Human Services, 1991).

In one embodiment the antibody comprises a VH domain having the sequence according to SEQ ID NO. 1. In another embodiment the antibody comprises a VL domain having the sequence according to SEQ ID NO. 2. Thus the antibody may comprise a VH domain and a VL domain where the VH comprises the sequence of SEQ ID NO.1 and the VL domain comprises the sequence of SEQ ID NO.2.

The VH and VL domain(s) in various embodiments form an antibody antigen binding site that binds PSMA.

In some embodiments the antibody is an intact antibody comprising a VH domain and a VL domain, the VH and VL domains having sequences of SEQ ID NO.1 paired with SEQ ID NO.2.

In one embodiment the light chain is a human kappa light chain.

In one embodiment, the antibody that binds to PSMA comprises a heavy chain with the amino acid sequence shown in SEQ ID NO: 9 and a light chain with the amino acid sequence shown in SEQ ID NO: 10.

As used herein, “binds PSMA” is used to mean that the cell binding agent or the antibody binds PSMA with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds PSMA with an association constant (Kₐ) at least 2, 3, 4, 5, 10, 20, 50, 100, 200, 500, 1000, 2000, 5000, 10⁴, 10⁵ or 10⁶-fold higher than the antibody's association constant for BSA, when measured at physiological conditions. The cell binding agents or antibodies of the disclosure can in some embodiments bind PSMA with a high affinity. For example, in some embodiments the antibody can bind PSMA with a K_{D} equal to or less than about 10⁻⁶ M, such as 1×10⁻⁶, 10⁻⁷, 10⁻⁸, 10⁻⁹, 10⁻¹⁰, 10⁻¹¹, 10⁻¹², 10⁻¹³ or 10⁻¹⁴.

As used herein, PSMA refers to Prostate-Specific Membrane Antigen. In one embodiment, PSMA polypeptide corresponds to Genbank accession no. AAA60209, version no. AAA60209.1 GI: 190664, record update date: Jun. 23, 2010 08:48 AM. In one embodiment, the nucleic acid encoding PSMA polypeptide corresponds to Genbank accession no. M99487, version no. M99487.1 GI: 190663, record update date: Jun. 23, 2010 08:48 AM.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method or may be made by recombinant DNA methods. The monoclonal antibodies may also be isolated from phage antibody libraries or from transgenic mice carrying a fully human immunoglobulin system.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567). Chimeric antibodies include “primatized” antibodies comprising variable domain antigen-binding sequences derived from a non-human primate and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

### Modification of Antibodies

The antibodies disclosed herein may be modified. For example, to make them less immunogenic to a human subject. This may be achieved using any of a number of techniques familiar to the person skilled in the art. Such techniques includes humanisation to reduce the in vivo immunogenicity of a non-human antibody or antibody fragment. There are a range of humanisation techniques, including ‘CDR grafting’, ‘guided selection’, ‘deimmunization’, ‘resurfacing’ (also known as ‘veneering’), ‘composite antibodies’, ‘Human String Content Optimisation’ and framework shuffling.

Other sequence modification can be made to assist with conjugation of drugs or other substances of interest to particular sites in the antibody or to regulate the drug to antibody ratio. For example one or more cysteine residues, such as in the hinge region, may be substituted or introduced, where conjugation to a cysteine residue is desired.

In one embodiment, the antibody has one or more cysteine residues introduced outside the hinge region, e.g. to take advantage of the THIOMAB™ site-specific approach (see Adhikari, P., Zacharias, N., Ohri, R., Sadowsky, J. (2020). Site-Specific Conjugation to Cys-Engineered THIOMAB™ Antibodies. In: Tumey, L. (eds) Antibody-Drug Conjugates. Methods in Molecular Biology, vol 2078. Humana, New York, NY; and Junutula et al., 2008, Site-specific conjugation of a cytotoxic drug to an antibody improves the therapeutic index. Nat Biotechnol 26(8):925-932. A particular example, used herein, is a substitution at amino acid residue 205 (EU numbering) of the IgG light chain, e.g. a V205C mutation.

In one embodiment, a heavy chain hinge region cysteine is mutated to remove a potential site for conjugation. A wild-type antibody typically has 8 surface-exposed cysteine residues available for conjugation—three in each of the heavy chain hinges (C220, C226 and C229—EU numbering and one at the C-terminus of each of the light chains (C214 kappa or C213 lambda—EU numbering).

The amino acid substituted for a cysteine residue can, for example, be selected from valine, serine, threonine, alanine, glycine, leucine and isoleucine. In one embodiment the amino acid substitution is a valine for the interchain cysteine residue.

Other modifications include those in the Fc region that reduce effector functions, such as Fc gamma binding (Wang et al., 2018, Protein Cell 9(1):63-73). For example, the sequence may include a PALALA series of mutations (L234A/L235A (Xu et al., 2000, Cell. Immunol. 200:16-26); and P329A (Michaelsen et al., 2009, Scand. J. Immunol. 70(6): 553-64)—EU numbering. Another example is an FES mutation (see Oganesyan et al., 2008, Acta Crystallographica, D64:700-704).

In one embodiment the antibody has an Fc region as shown in SEQ ID NO: 9.

### Exatecan Warhead

Exatecan is a topoisomerase inhibitor. It is a more extensively modified derivative of camptothecin, with an additional alicyclic ring fused to rings A and B that bears a solubilising primary amine. There are also lipophilic substituents at positions 10 and 11 on ring A that may help to enhance membrane permeability. It is about 2.5-fold more potent than SN38 (the active metabolite of irinotecan) at stabilizing the topo I/DNA complex, and as a cytotoxin in a variety of cell lines.

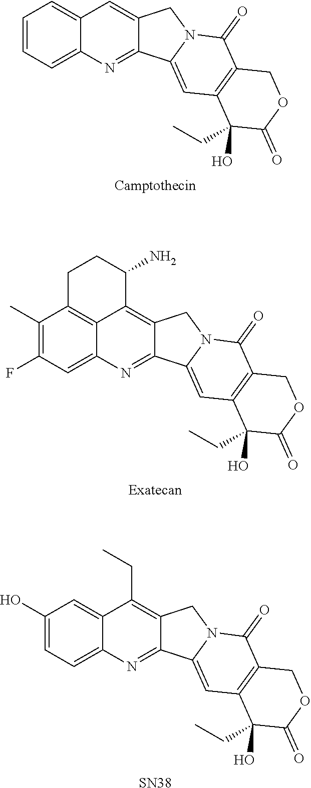 [SMILES: CC[C@@]1(O)C(=O)OCc2c1cc1n(c2=O)Cc2cc3ccccc3nc2-1 ; CC[C@@]1(O)C(=O)OCc2c1cc1n(c2=O)Cc2c-1nc1cc(F)c(C)c3c1c2[C@@H](N)CC3 ; CCc1c2c(nc3ccc(O)cc13)-c1cc3c(c(=O)n1C2)COC(=O)[C@]3(O)CC ; "SN38"]

Exatecan (also known as DX-8951) is available from a number of commercial sources e.g. as Exatecan mesylate (CAS No.: 169869-90-3).

### Drug Linkers

A wide variety of linker technologies are available in the art to link cytotoxins to cell binding agents. Linkers can incorporate various different moieties to assist with antibody-drug conjugate stability and determine drug release characteristics. For example the linker may include a cleavable moiety, such as one that is cleavable by cathepsin B (e.g. Valine-Alanine or Valine-Citrulline).

The functionality that allows conjugation to the cell binding agent is based on the site of conjugation and its chemistry. For conjugation to cysteines, thiol-reactive maleimide is the most applied reactive handle, although it is also possible to create a disulfide bridge by oxidation with a linker bearing a sulfhydryl group. Aldehyde or keto functional groups such as oxidized sugar groups or pAcPhe unnatural amino acids can be reacted with hydrazides and alkoxyamines to yield acid-labile hydrazones or oxime bonds. In addition, a hydrazine can be coupled with an aldehyde via HIPS ligation to generate a stable C-C linkage.

In some embodiments, the antibody drug conjugates of the disclosure can be described as Ab-L-D, where Ab is the anti-PSMA antibody, D is Exatecan and L is a linker. The number of Drug moieties per Ab (the drug loading, p) depends on the number of linkers attached to each Ab, and the number of Drug moieties per linker. Typically the drug loading, p, is from 1 to 8, such as from 1 to 2, 1 to 4, 1 to 6, 2 to 6 or 3 to 6, such as from 3 to 6. Where site-specific approaches are used, such as the N-linked glycosylation site at Asn-297, conjugation via a glutamine residue using microbial transglutaminase or where mutations have been made to reduce the number of endogenous cysteine sites available for conjugation, the number of sites available for conjugation may be limited to two per antibody. In some embodiments one Drug moiety is joined to each linker whereas in others, more than one Drug moiety may be joined to each linker (e.g. a branched linker with two or more Drug moieties per linker)

Drug loading is typically considered on an average basis since variations can arise from the conjugation process (a composition comprising a plurality of antibody drug conjugate molecules will typically have individual molecules with from zero to the maximum number of drug molecules possible). Methods for determining average drug loading are known in the art.

The linker comprises a polyglycine stretch, Glyₙ where n is from 5 to 8 glycines, which we have shown is beneficial in reducing aggregation. The glycines are continuous e.g. Gly-Gly-Gly-Gly-Gly, with no intervening amino acids. In one embodiment n is 5 or 6, such as 5.

The drug linker is designed so that under cellular conditions, the Exatecan warhead is released without any remaining portions of the linker (e.g. by the use of self-immolative linker chemistry, such as para-aminobenzylcarbamate—PABC).

In one embodiment the linker is of formula (la):-G^{LL}-X-Glyₙ-A-

wherein G^{LL} is for connection of the linker to a cysteine residue on the antibody; X is an optional spacer portion; n is from 5 to 8; and A comprises a moiety cleavable under cellular conditions and optionally a self-immolative moiety.

### G^{LL}

G^{LL} may be selected from:

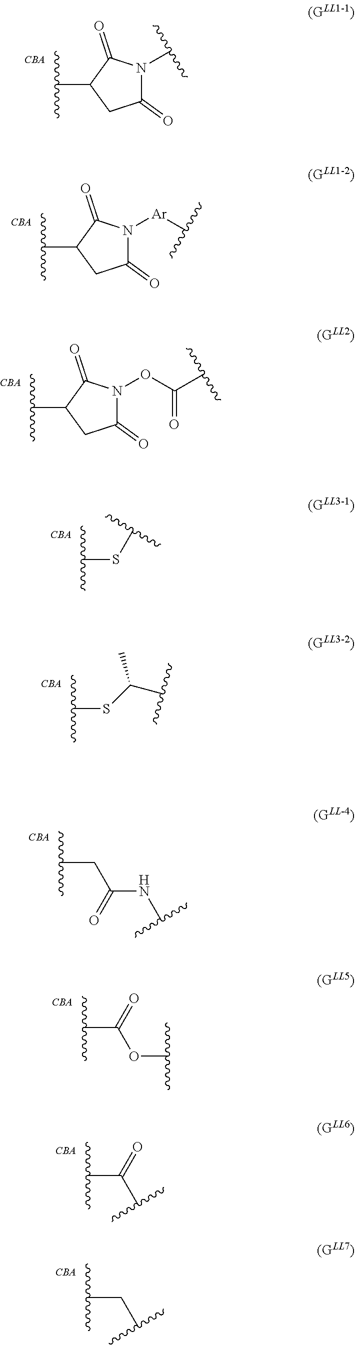

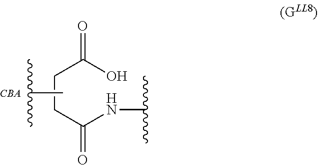

where Ar represents a C₅₋₆ arylene group, e.g. phenylene and X represents C₁₋₅ alkyl. CBA indicates the end of G^{LL} connected to the antibody.

In some embodiments, G^{LL} is selected from G^{LL1-1} and G^{LL1-2}. In some of these embodiments, G^{LL} is G^{LL1-1}.

C₅₋₆ arylene: The term “C₅₋₆ arylene”, as used herein, pertains to a divalent moiety obtained by removing two hydrogen atoms from an aromatic ring atom of an aromatic compound.

In this context, the prefixes (e.g. C₅₋₆) denote the number of ring atoms, or range of number of ring atoms, whether carbon atoms or heteroatoms.

The ring atoms may be all carbon atoms, as in “carboarylene groups”, in which case the group is phenylene (C₆).

Alternatively, the ring atoms may include one or more heteroatoms, as in “heteroarylene groups”. Examples of heteroarylene groups include, but are not limited to, those derived from:
N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆); and
N₃: triazole (C₅), triazine (C₆).

C₁₋₅ alkyl: The term “C₁₋₅ alkyl” as used herein, pertains to a monovalent moiety obtained by removing a hydrogen atom from a carbon atom of a hydrocarbon compound having from 1 to 5 carbon atoms, which may be aliphatic or alicyclic, and which may be saturated or unsaturated (e.g. partially unsaturated, fully unsaturated). The term “C₁₋₅ alkyl” as used herein, pertains to a monovalent moiety obtained by removing a hydrogen atom from a carbon atom of a hydrocarbon compound having from 1 to n carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

### X

X is an optional linker portion connecting G^{LL} to the polyglycine stretch. Where X is present, X may for example be selected from —(CR¹R²)y—, —(CR¹R²)y—C(O)—, and —C(O)—(CR¹R²)y—C(O)—, wherein y is an integer in the range 0 to 6, wherein one or more CR¹R² may be optionally replaced by NR¹; R¹ and R² are independently selected from H, R^{2a}, C(O)OH and C(O)R^{2a}, wherein R^{2a} is selected from optionally substituted C₁-C₆ (hetero) alkyl groups, C₃-C₁₀ (hetero) cycloalkyl groups, C₆-C₁₀ (hetero) aryl groups, C₇-C₁₄ alkyl (hetero)aryl groups and C₇-C₁₄ (hetero) arylalkyl groups; wherein R^{3a} is independently selected from hydrogen and C₁-C₄ alkyl groups.

In one embodiment, y is an integer in the range 1 to 6, such as from 1 to 5, 1 to 4, 1 to 3, or 1 or 2.

In one embodiment R¹ and R² are both H.

In one embodiment, X is —(CH₂)₁₋₅—C(O)—. In a particular embodiment X is —CH₂C(O)— or —CH₂CH₂C(O)—.

### A

Linker portion A connects the polyglycine stretch to the Exatecan and includes components that allow for cleavage of the linker under cellular conditions e.g. cellular protease recognition motifs such as a cleavage site for cathepsin.

For example A may comprise a dipeptide selected from:
^{NH}-Phe-Lys-^{C═O}, ^{NH}-Val-Ala-^{C═O}, ^{NH}-Val-Lys-^{C═O}, ^{NH}-Ala-Lys-^{C═O}, ^{NH}-Val-Cit-^{C═O},
^{NH}-Phe-Cit-^{C═O}, NH-Leu-Cit-^{C═O}, ^{NH}-Ile-Cit-^{C═O}, ^{NH}-Phe-Arg-^{C═O}, ^{NH}-Trp-Cit-^{C═O}, and
^{NH}-Gly-Val-^{C═O};
   where Cit is citrulline.

In one embodiment the dipeptide is Val-Ala or Val-Cit, such as Val-Ala.

In the above representations of dipeptide residues, NH-represents the N-terminus, and —C═O represents the C-terminus of the residue. The N-terminus is orientated towards the polyglycine stretch.

The self-immolative moiety which allows for the generation of a free amine on the Exatecan F ring is typically p-aminobenzylcarbamate (PABC) or a variant thereof. Accordingly in one embodiment, A is Val-Cit-PABC or Val-Ala-PABC.

In one embodiment the linker is of formula (IIa):

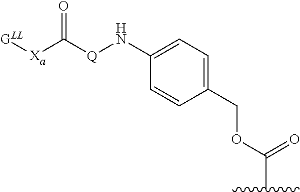

wherein G^{LL} is as defined above, and Xₐ is selected from —(CR¹R²)y— and —C(O)—(CR¹R²)y—, wherein y is an integer in the range 0 to 6, wherein one or more CR¹R² may be optionally replaced by NR¹; R¹ and R² are independently selected from H, R^{2a}, C(O)OH and C(O)R^{2a}, wherein R^{2a} is selected from optionally substituted C₁-C₆ (hetero) alkyl groups, C₃-C₁₀ (hetero)cycloalkyl groups, C₆-C₁₀ (hetero)aryl groups, C₇-C₁₄ alkyl(hetero)aryl groups and C₇-C₁₄ (hetero)arylalkyl groups; wherein R^{3a} is independently selected from hydrogen and C₁-C₄ alkyl groups.

In one embodiment, y is an integer in the range 1 to 6, such as from 1 to 5, 1 to 4, 1 to 3, or 1 or 2.

In one embodiment R¹ and R² are both H.

In one embodiment, Xₐ is —(CH₂)₁₋₅—. In a particular embodiment Xₐ is —CH₂— or —CH₂CH₂—.

In a particular embodiment G^{LL} is G^{LL1-1}

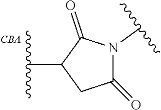 [SMILES: *C1CC(=O)N(*)C1=O]

Q is-Glyₙ-AA₁-AA₂ where n is from 5 to 8, such as 5 or 6 and AA₁-AA₂ is a dipeptide residue which is a recognition site for cathepsin such that the linker is susceptible to cathepsin-mediated cleavage.

In one embodiment, AA₁-AA₂ is selected from:
^{NH}-Phe-Lys-^{C═O}, ^{NH}-Val-Ala-^{C═O}, ^{NH}-Val-Lys-^{C═O}, ^{NH}-Ala-Lys-^{C═O}, ^{NH}-Val-Cit-^{C═O},
^{NH}-Phe-Cit-^{C═O}, NH-Leu-Cit-^{C═O}, ^{NH}-Ile-Cit-^{C═O}, ^{NH}-Phe-Arg-^{C═O}, ^{NH}-Trp-Cit-^{C═O}, and
^{NH}-Gly-Val-^{C═O};
   where Cit is citrulline.

In one embodiment the dipeptide is Val-Ala or Val-Cit, such as Val-Ala.

In the above representations of dipeptide residues, NH- represents the N-terminus, and —C═O represents the C-terminus of the residue. The C-terminus binds to the NH attached to the benzene ring.

The linker is typically connected to the Exatecan via the NH₂ on the F ring.

In any of the above embodiments, the linker may include a branched structure such that there is at least one Exatecan drug moiety conjugated to each branch.

### Drug Linker Embodiments

In some embodiments, the drug linker, L-D, is

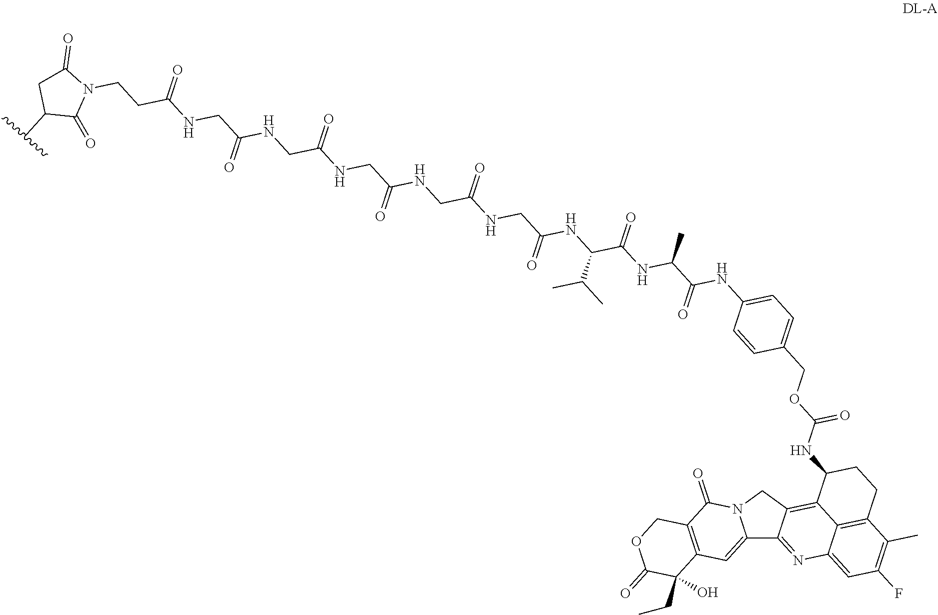 [SMILES: *C1CC(=O)N(CCC(=O)NCC(=O)NCC(=O)NCC(=O)NCC(=O)NCC(=O)N[C@H](C(=O)N[C@@H](C)C(=O)Nc2ccc(COC(=O)N[C@H]3CCc4c(C)c(F)cc5nc6c(c3c45)Cn3c-6cc4c(c3=O)COC(=O)[C@]4(O)CC)cc2)C(C)C)C1=O]

A preferred antibody drug conjugate has the drug linker above linked to a PSMA antibody with a heavy chain as shown in SEQ ID NO: 9 and a light chain as shown in SEQ ID NO: 10.

### Site of Conjugation and Drug Loading

Drug linkers can be conjugated to a cell binding agent, such as an antibody, using a variety of methods known in the art and at a number of different sites. Conjugation sites may be cysteine residues in the antibody sequence (endogenous or engineered). With respect to cysteine conjugation, in one embodiment the cysteine is an endogenous cysteine located in the hinge region or Fc domain or a cysteine in the constant region of the light chain (for example C220, C226 or C229 of the heavy chain in IgG1 or the equivalent; or C214 (kappa) or C213 (lambda) of the light chain (EU numbering)). In another embodiment the cysteine is an engineered cysteine introduced in the hinge region or constant region of the light chain or heavy chain.

The drug loading is the average number of Exatecan drugs per cell binding agent, e.g. antibody, in a composition comprising a plurality of molecules.

The average number of drugs per antibody in preparations of ADC from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al (2004) Clin. Cancer Res. 10:7063-7070; Sanderson et al (2005) Clin. Cancer Res.

11:843-852). However, the distribution of p (drug) values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous ADC where p is a certain value from ADC with other drug loadings may be achieved by means such as reverse phase HPLC or electrophoresis. Such techniques are also applicable to other types of conjugates.

For some antibody drug conjugates, p may be limited by the number of attachment sites on the antibody. For example, an antibody may have only one or several cysteine thiol groups, or may have only one or several sufficiently reactive thiol groups through which a linker may be attached. Higher drug loading, e.g. p>5, may cause aggregation, insolubility, toxicity, or loss of cellular permeability of certain antibody-drug conjugates.

Typically, fewer than the theoretical maximum of drug moieties are conjugated to an antibody during a conjugation reaction. Only the most reactive cysteine thiol groups may react with a thiol-reactive linker reagent. Generally, antibodies do not contain many, if any, free and reactive cysteine thiol groups which may be linked to a drug moiety. Most cysteine thiol residues in the antibodies of the compounds exist as disulfide bridges and must be reduced with a reducing agent such as dithiothreitol (DTT) or TCEP, under partial or total reducing conditions. The loading (drug/antibody ratio) of an ADC may be controlled in several different manners, including: (i) limiting the molar excess of drug-linker intermediate or linker reagent relative to antibody, (ii) limiting the conjugation reaction time or temperature, and (iii) partial or limiting reductive conditions for cysteine thiol modification.

Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by engineering one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

Cysteine amino acids may be engineered at reactive sites in an antibody, and which do not form intrachain or intermolecular disulfide linkages (Junutula, et al., 2008b Nature Biotech., 26(8):925-932; Dornan et al. (2009) Blood 114(13):2721-2729; U.S. Pat. Nos. 7,521,541; 7,723,485; WO2009/052249). The engineered cysteine thiols may react with linker reagents or the drug-linker reagents of the present disclosure which have thiol-reactive, electrophilic groups such as maleimide or alpha-halo amides to form ADC with cysteine engineered antibodies and the Exatecan drug moieties. The location of the drug moiety can thus be designed, controlled, and known. The drug loading can be controlled since the engineered cysteine thiol groups typically react with thiol-reactive linker reagents or drug-linker reagents in high yield. Engineering an IgG antibody to introduce a cysteine amino acid by substitution at a single site on the heavy or light chain gives two new cysteines on the symmetrical antibody. This THIOMAB™ approach allows for site-specific conjugation with more homogenous DARs. In one embodiment, a single engineered cysteine residue is introduced and the resulting maximum DAR is 2 (in a composition comprising a mixture, p is from 1.5 to 2).

Conversely, one or more of the endogenous cysteine residues may be mutated to reduce the number of available conjugation sites (as described in the “Modification of antibodies” section above). Since p>5 or 6 can lead to undesirable aggregation, where all four hinge cysteines are available (a total of 8), it can be necessary to tailor the conjugation process to avoid high levels of conjugation as this can lead to significant numbers of species where p=7 or 8. However, avoiding this results in a lower overall average drug to antibody ratio (e.g. around 4). To push the DAR higher whilst avoiding high p ratios can be achieved according to the present disclosure by mutating one of the hinge cysteine residues to leave 2 available conjugation sites per heavy chain and one per light chain—6 in total. In this way a DAR closer to 6 can be achieved whilst avoiding species where p=7 or 8 which may have a tendency to aggregate.

Where more than one nucleophilic or electrophilic group of the antibody reacts with a drug-linker intermediate, or linker reagent followed by drug moiety reagent, then the resulting product is a mixture of ADC compounds with a distribution of drug moieties attached to an antibody, e.g. 1, 2, 3, etc. Liquid chromatography methods such as polymeric reverse phase (PLRP) and hydrophobic interaction (HIC) may separate compounds in the mixture by drug loading value. Preparations of ADC with a single drug loading value (p) may be isolated, however, these single loading value ADCs may still be heterogeneous mixtures because the drug moieties may be attached, via the linker, at different sites on the antibody.

Thus the antibody-drug conjugate compositions of the disclosure include mixtures of antibody-drug conjugate compounds where the antibody has one or more Exatecan drug moieties and where the drug moieties may be attached to the antibody at various amino acid residues.

In one embodiment, the average number of Exatecan groups per antibody is in the range 1 to 8. In some embodiments the range is selected from 1 to 2, 1 to 4, 1 to 6, 2 to 6 or 3 to 6, such as from 1 to 2 or 3 to 6.

### Preparation of Drug Conjugates

The drug linker may be synthesised as described in, for example, the experimental section below.

The antibody drug conjugates of the present disclosure may then be prepared by conjugating the drug-linker to the antibody via an endogenous or engineered cysteine residue via maleimide, as for example described in U.S. Pat. No. 9,889,207 or Flynn et al., 2016. Mol Cancer Ther 15:2709.

### Treated Disorders

The therapies described herein include those with utility for anti-cancer activity. In particular, in certain aspects the therapies include an antibody conjugated, i.e. covalently attached by a linker, to an Exatecan drug moiety, i.e. toxin. When the drug is not conjugated to an antibody, the Exatecan drug has a cytotoxic effect. The biological activity of the Exatecan drug moiety is thus modulated by conjugation to an antibody. The antibody-drug conjugates (ADC) of the disclosure selectively deliver an effective dose of a cytotoxic agent to tumor tissue whereby greater selectivity, i.e. a lower efficacious dose, may be achieved.

Thus, in one aspect, the present disclosure provides therapies comprising administering a conjugate compound as described herein, which binds to PSMA, for use in therapy, wherein the method comprises selecting a subject based on expression of PSMA protein.

In one aspect, the present disclosure provides a therapy with a label that specifies that the therapy is suitable for use with a subject determined to be suitable for such use. The label may specify that the therapy is suitable for use in a subject has expression of PSMA e.g. is a PSMA+ve cancer. The label may specify that the subject has a particular type of cancer.

The label may specify that the subject has a PSMA+ve cancer.

The range of disorders that may be treated by such therapies is described in more detail below.

In a further aspect there is also provided a therapy as described herein for use in the treatment of a proliferative disease. Another aspect of the present disclosure provides the use of a conjugate compound as described herein in the manufacture of a medicament for treating a proliferative disease.

One of ordinary skill in the art is readily able to determine whether or not a candidate therapy treats a proliferative condition for any particular cell type. For example, assays which may conveniently be used to assess the activity offered by a particular compound are described below.

The therapies described herein may be used to treat a proliferative disease. The term “proliferative disease” pertains to an unwanted or uncontrolled cellular proliferation of excessive or abnormal cells which is undesired, such as, neoplastic or hyperplastic growth, whether in vitro or in vivo.

The proliferative disease may be characterised by the presence of a neoplasm comprising both PSMA+ve and PSMA−ve cells.

The target neoplasm or neoplastic cells may be all or part of a solid tumour, such as an advanced solid tumour.

Thus in one embodiment the neoplasm/cancer is itself essentially PSMA+ve. This includes prostate cancer.

In addition, previous studies have found that PSMA is upregulated on the endothelial cells of the neovasculature of a wide variety of solid tumors where it may facilitate endothelial cell sprouting and invasion through its regulation of lytic proteases that have the ability to cleave the extracellular matrix (Van de Wiele et al., 2020, Histopathol. 35(9): 919:927). Accordingly, conjugate compounds described herein may be used to treat solid tumours by targeting PSMA expressed in the neovasculature of such tumours.

Thus examples of proliferative conditions that may be treated with the conjugate compounds described herein include, but are not limited to, benign, pre-malignant, and malignant cellular proliferation, including but not limited to, neoplasms, tumours and cancers, such as histocytoma, glioma, glioblastoma, astrocyoma, osteoma, lung cancer, small cell lung cancer, non-small cell lung cancer, gastrointestinal cancer, gastric cancer, bowel cancer, colon cancer, colorectal cancer, hepatocellular carcinoma, breast cancer, ovarian cancer, prostate cancer, testicular cancer, liver cancer, kidney/renal cancer, bladder cancer, pancreatic cancer, brain cancer, head and neck cancer, thyroid cancer, neuroblastoma, neuroendocrine cancer, sarcoma, osteosarcoma, Kaposi's sarcoma, squamous cell carcinoma, melanoma, and lymphomas.

In a particular embodiment, the condition is selected from renal cell cancer, bladder transitional cell carcinoma, colonic adenocarcinoma, hepatocellular carcinoma, neuroendocrine carcinoma, glioblastoma, melanoma, pancreatic cancer, such as pancreatic duct carcinoma, soft tissue sarcoma, ovarian cancer, endometrial cancer, breast cancer, colorectal, gastric and lung cancer such as non-small cell lung carcinoma, mesothelioma. PSMA is positive in the neovasculature of these tumours.

Prostate cancer (such as hormone-sensitive, metastatic prostate cancer, mHSPC), adenoid cystic carcinoma of the head and neck, and thyroid cancer are cancers of particular interest.

### Patient Selection

In certain aspects, the individuals are selected as suitable for treatment with the treatments before the treatments are administered.

As used herein, individuals who are considered suitable for treatment are those individuals who are expected to benefit from, or respond to, the treatment. Individuals may have, or be suspected of having, or be at risk of having cancer. Individuals may have received a diagnosis of cancer. Typically the individual is an animal or human subject.

In some aspects, individuals are selected on the basis of the amount or pattern of expression of a first target protein. In some aspects, the selection is based on expression of a first target protein at the cell surface.

In some cases, individuals are selected on the basis they have, or are suspected of having, are at risk of having cancer, or have received a diagnosis of a proliferative disease characterised by the presence of a neoplasm comprising cells having a high level of surface expression of PSMA. The neoplasm may be composed of cells having a high level of surface expression of PSMA. In some cases, high levels of surface expression means that mean number of anti-PSMA antibodies bound per neoplastic cell is greater than 70000, such as greater than 80000, greater than 90000, greater than 100000, greater than 110000, greater than 120000, greater than 130000, greater than 140000, or greater than 150000.

In some cases, individuals are selected on the basis they have, or are suspected of having, are at risk of having cancer, or have received a diagnosis of a proliferative disease characterised by the presence of a neoplasm comprising cells having a low level of surface expression of PSMA. The neoplasm may be composed of cells having a low level of surface expression of PSMA. In some cases, low levels of surface expression means that mean number of anti-PSMA antibodies bound per neoplastic cell is less than 20000, such as less than 80000, less than 70000, less than 60000, less than 50000, less than 40000, less than 30000, less than 20000, less than 10000, or less than 5000.

In some aspects, individuals are selected on the basis they have a neoplasm comprising both PSMA+ve and PSMA−ve cells. The neoplasm may be composed of PSMA−ve neoplastic cells, optionally wherein the PSMA−ve neoplastic cells are associated with PSMA+ve neoplastic or non-neoplastic cells. The neoplasm or neoplastic cells may be all or part of a solid tumour. The solid tumour may be partially or wholly PSMA−ve.

In some cases, expression of PSMA in a particular tissue of interest is determined. For example, in a sample of tumor tissue. In some cases, systemic expression of the target is determined. For example, in a sample of circulating fluid such as blood, plasma, serum or lymph.

In some aspects, the individual is selected as suitable for treatment due to the presence or absence of PSMA expression in a sample. In those cases, individuals without PSMA expression may be considered not suitable for treatment.

In other aspects, the level of PSMA expression is used to select a individual as suitable for treatment. Where the level of expression of PSMA is above a threshold level, the individual is determined to be suitable for treatment.

In some aspects, the presence of PSMA in cells in the sample indicates that the individual is suitable for treatment with an ADC as disclosed herein. In other aspects, the amount of PSMA expression must be above a threshold level to indicate that the individual is suitable for treatment. In some aspects, the observation that PSMA localisation is altered in the sample as compared to a control indicates that the individual is suitable for treatment.

In some aspects, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express a first target protein. In some aspects disclosed herein, a patient is determined to be suitable for treatment if at least at least 10% of the cells in the sample express a first target protein.

In some aspects, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express a second target protein. In some aspects disclosed herein, a patient is determined to be suitable for treatment if at least at least 10% of the cells in the sample express a second target protein.

In some aspects the sample is taken from a bodily fluid, more preferably one that circulates through the body. Accordingly, the sample may be a blood sample or lymph sample. In some cases, the sample is a urine sample or a saliva sample.

In some cases, the sample is a blood sample or blood-derived sample. The blood derived sample may be a selected fraction of a individual's blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC). Accordingly, methods according to the present disclosure may involve detection of a first target polypeptide or nucleic acid in the blood, in white blood cells, peripheral blood mononuclear cells, granulocytes and/or red blood cells.

In another aspect the sample is a biopsy of solid tissue e.g. one that could include the neovasculature of a solid tumour if present.

The sample may be fresh or archival. For example, archival tissue may be from the first diagnosis of an individual, or a biopsy at a relapse. In certain aspects, the sample is a fresh biopsy.

The terms “subject”, “patient” and “individual” are used interchangeably herein.

In some aspects disclosed herein, an individual has, or is suspected as having, or has been identified as being at risk of, a proliferative disease such as cancer. In some aspects disclosed herein, the individual has already received a diagnosis of such a disease. A list of relevant diseases is provided above in the section “Treated disorders”. Ovarian cancer, non-small cell lung carcinoma (NSCLC), gastric cancer, oesophageal cancer, endometrial cancer and hepatocellular carcinoma (HCC) are conditions of particular interest.

In some cases, the individual has received a diagnosis of a proliferative disease such as cancer, such as one of the disorders listed above. Ovarian cancer, non-small cell lung carcinoma (NSCLC), gastric cancer, oesophageal cancer, endometrial cancer and hepatocellular carcinoma (HCC) are conditions of particular interest.

In some cases, the individual has received a diagnosis of a solid cancer containing PSMA+expressing cells.

The individual may be undergoing, or have undergone, a therapeutic treatment for that cancer. The subject may, or may not, have previously received an anti-PSMA ADC. In some cases the cancer is prostate cancer.

### Methods of Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount” or “effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Disclosed herein are methods of therapy. Also provided is a method of treatment, comprising administering to a subject in need of treatment a therapeutically-effective amount of an ADC. The term “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors. The subject may have been tested to determine their eligibility to receive the treatment according to the methods disclosed herein. The method of treatment may comprise a step of determining whether a subject is eligible for treatment, using a method disclosed herein.

The treatment may involve administration of the ADC alone or in combination with other treatments, either simultaneously or sequentially dependent upon the condition to be treated. Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Also included in the definition of “chemotherapeutic agent” are: (i) anti-hormonal agents that act to regulate or inhibit hormone action on tumors such as anti-estrogens and selective estrogen receptor modulators (SERMs); (ii) aromatase inhibitors that inhibit the enzyme aromatase, which regulates estrogen production in the adrenal glands; (iii) anti-androgens; (iv) protein kinase inhibitors such as MEK inhibitors; (v) lipid kinase inhibitors; (vi) anti-angiogenic agents.

Also included in the definition of “chemotherapeutic agent” are therapeutic antibodies, including bispecific antibodies.

Compositions according to the present disclosure are preferably pharmaceutical compositions. Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which will typically be by injection, e.g. cutaneous, subcutaneous, or intravenous.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

### Dosage

It will be appreciated by one of skill in the art that appropriate dosages of the ADC, and compositions comprising these active elements, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In certain aspects, the dosage of ADC is determined by the expression of PSMA observed in a sample obtained from the subject. Thus, the level or localisation of expression of PSMA in the sample may be indicative that a higher or lower dose of ADC is required. For example, a high expression level of PSMA may indicate that a higher dose of ADC would be suitable. In some cases, a high expression level of PSMA may indicate the need for administration of another agent in addition to the ADC. For example, administration of the ADC in conjunction with a chemotherapeutic agent. A high expression level of PSMA may indicate a more aggressive therapy.

In certain aspects, the dosage level is determined by the expression of PSMA on neoplastic cells in a sample obtained from the subject. For example, when the target neoplasm is composed of, or comprises, neoplastic cells expressing PSMA.

In certain aspects, the dosage level is determined by the expression of PSMA on cells associated with the target neoplasm. For example, the target neoplasm may be a solid tumour composed of, or comprising, neoplastic cells that express PSMA. For example, the target neoplasm may be a solid tumour composed of, or comprising, neoplastic cells that do not express PSMA. The cells expressing PSMA may be neoplastic or non-neoplastic cells associated with the target neoplasm.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

For the ADC, where it is an Exatecan bearing ADC, the dosage amounts described above may apply to the conjugate (including the Exatecan moiety and the linker to the antibody) or to the effective amount of Exatecan compound provided, for example the amount of compound that is releasable after cleavage of the linker.

### Specific Embodiments

Embodiment 1—An antibody drug conjugate of formula (I): Ab-L-Dp, wherein:
Ab is an antibody that binds to PSMA, which antibody comprises (i) an immunoglobulin heavy chain variable region having a CDR1 region with the amino acid sequence shown in SEQ ID NO: 3, a CDR2 region with the amino acid sequence shown in SEQ ID NO: 4, and a CDR3 region with the amino acid sequence shown in SEQ ID NO: 5; and (ii) an immunoglobulin light chain variable region having a CDR1 region with the amino acid sequence shown in SEQ ID NO: 6, a CDR2 region with the amino acid sequence shown in SEQ ID NO: 7, and a CDR3 region with the amino acid sequence shown in SEQ ID NO: 8;
L-Dp is a drug linker conjugated to the Ab via one or more cysteine residues, wherein L comprises a polyglycine moiety, Glyn wherein n is from 5 to 8, and D is an Exatecan, wherein the drug linker can be cleaved under cellular conditions to release an Exatecan with the following formula (II):

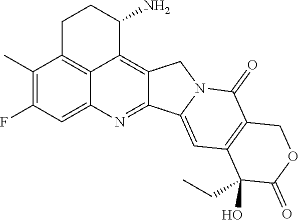 [SMILES: CC[C@@]1(O)C(=O)OCc2c1cc1n(c2=O)Cc2c-1nc1cc(F)c(C)c3c1c2[C@@H](N)CC3]

and wherein p is the number of drug units per antibody and is from 1 to 8, such as 1 to 6.

Embodiment 2—A conjugate according to embodiment 1 wherein the antibody has a VH domain as shown in SEQ ID NO: 1.

Embodiment 3—A conjugate according to embodiment 1 or embodiment 2 wherein the antibody has a VL domain as shown in SEQ ID NO: 2.

Embodiment 4—A conjugate according to any one of embodiments 1 to 3 wherein the antibody has a heavy chain sequence as shown in SEQ ID NO: 9 and/or a light chain sequence as shown in SEQ ID NO: 10.

Embodiment 5—A conjugate according to any one of the preceding embodiments wherein the linker comprises -G^{LL}-X-Glyₙ-A-wherein G^{LL} is for connection of the linker to a cysteine residue on the antibody and is as defined herein; X is an optional spacer portion as defined herein; n is from 5 to 8; and A comprises a moiety cleavable under cellular conditions and optionally a self-immolative moiety.

Embodiment 7—A conjugate according to any one of the preceding embodiments wherein the linker comprises a cathepsin cleavable sequence e.g. Val-Ala or Val-Cit.

Embodiment 8—A conjugate according to any one of the preceding embodiments wherein the linker comprises a self-immolative moiety operably linked to the Exatecan, such as para-aminobenzylcarbamate.

Embodiment 9—A conjugate according to any one of the preceding embodiments where L-Dp is:

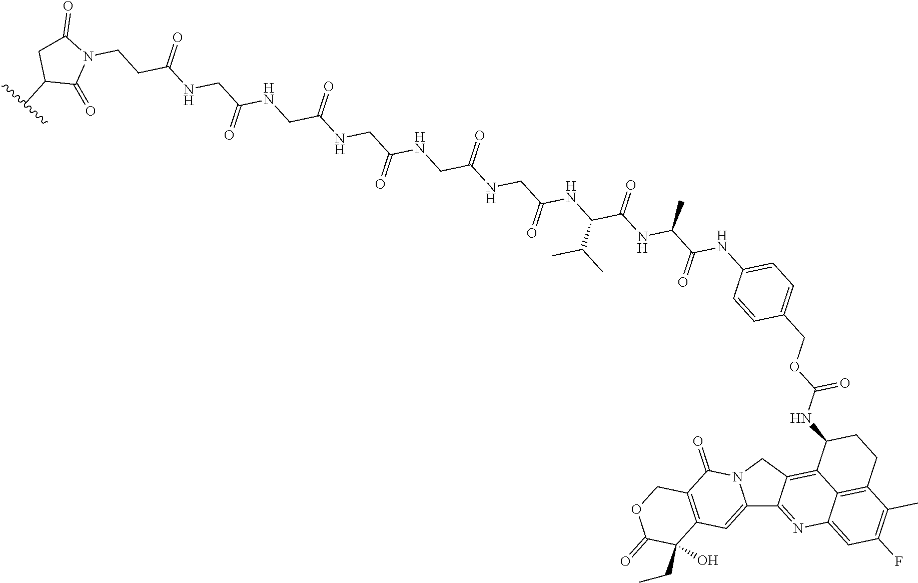 [SMILES: *C1CC(=O)N(CCC(=O)NCC(=O)NCC(=O)NCC(=O)NCC(=O)NCC(=O)N[C@H](C(=O)N[C@@H](C)C(=O)Nc2ccc(COC(=O)N[C@H]3CCc4c(C)c(F)cc5nc6c(c3c45)Cn3c-6cc4c(c3=O)COC(=O)[C@]4(O)CC)cc2)C(C)C)C1=O]

Embodiment 10—A composition comprising a mixture of antibody drug conjugates according to any one of embodiments 1 to 11 wherein the average drug to antibody ratio is from 3 to 6.

Embodiment 11—A pharmaceutical composition comprising a conjugate or composition according to any one of the previous embodiments together with one or more pharmaceutically acceptable carriers or diluents, such as a pharmaceutical composition in liquid or lyophilized form.

Embodiment 12—A method of treating an individual suffering from a proliferative disease, for example a disease characterized by over-expression of PSMA, which method comprises administering to the patient an antibody drug conjugate according to any one of the embodiments 1 to 9 or a composition according to embodiment 10 or embodiment 11.

Embodiment 12—A method of treating an individual patient suffering cancer from prostate cancer, adenoid cystic carcinoma of the head and neck, or thyroid cancer which method comprises administering to the individual an antibody drug conjugate according to any one of embodiments 1 to 9 or a composition according to embodiment 10 or embodiment 11.

### Sequence Listing Part of the Description

| [2A10 VH] | |
|---|---|
| SEQ ID NO. 1 | |
| EVQLVQSGAE VKKPGESLKI SCKGSGYSFT SNWIGWVRQM PGKGLEWMGI IYPGDSDTRY | 60 |
| SPSFQGQVTI SADKSISTAY LQWSSLKASD TAMYYCARQT GFLWSSDLWG RGTLVTVSS | 119 |
| [2A10 VL] | |
| SEQ ID NO. 2 | |
| AIQLTQSPSS LSASVGDRVT ITCRASQDIS SALAWYQQKP GKAPKLLIYD ASSLESGVPS | 60 |
| RFSGYGSGTD FTLTINSLQP EDFATYYCQQ FNSYPLTFGG GTKVEIK | 107 |
| [2A10 VH, Kabat CDR1] | |
| SEQ ID NO. 3 | |
| SNWIG | |
| [2A10 VH, Kabat CDR2] | |
| SEQ ID NO. 4 | |
| IIYPGDSDTR YSPSFQG | |
| [2A10 VH, Kabat CDR3] | |
| SEQ ID NO. 5 | |
| QTGFLWSSDL | |
| [2A10 VL, Kabat CDR1] | |
| SEQ ID NO. 6 | |
| RASQDISSAL A | |
| [2A10 VL, Kabat CDR2] | |
| SEQ ID NO. 7 | |
| DASSLES | |
| [2A10 VL, Kabat CDR3] | |
| SEQ ID NO. 8 | |
| QQFNSYPLT | |
| [2A10-Heavy chain] | |
| SEQ ID NO. 9 | |
| EVQLVQSGAE VKKPGESLKI SCKGSGYSFT SNWIGWVRQM PGKGLEWMGI IYPGDSDTRY | 60 |
| SPSFQGQVTI SADKSISTAY LqWSSLKASD TAMYYCARQT GFLWSSDLWG RGTLVTVSSA | 120 |
| STKGPSVFPL APSSKSTSGG TAALGCLVKD YFPEPVTVSW NSGALTSGVH TFPAVLQSSG | 180 |
| LYSLSSVVTV PSSSLGTQTY ICNVNHKPSN TKVDKKVEPK SCDKTHTCPP CPAPELLGGP | 240 |
| SVFLFPPKPK DTLMISRTPE VTCVVVDVSH EDPEVKFNWY VDGVEVHNAK TKPREEQYNS | 300 |
| TYRVVSVLTV LHQDWLNGKE YKCKVSNKAL PAPIEKTISK AKGQPREPQV YTLPPSREEM | 360 |
| TKNQVSLTCL VKGFYPSDIA VEWESNGQPE NNYKTTPPVL DSDGSFFLYS KLTVDKSRWQ | 420 |
| QGNVFSCSVM HEALHNHYTQ KSLSLSPG | 448 |
| [2A10-Light chain] | |
| SEQ ID NO. 10 | |
| AIQLTQSPSS LSASVGDRVT ITCRASQDIS SALAWYQQKP GKAPKLLIYD ASSLESGVPS | 60 |
| RFSGYGSGTD FTLTINSLQP EDFATYYCQQ FNSYPLTFGG GTKVEIKRTV AAPSVFIFPP | 120 |
| SDEQLKSGTA SVVCLLNNFY PREAKVQWKV DNALQSGNSQ ESVTEQDSKD STYSLSSTLT | 180 |
| LSKADYEKHK VYACEVTHQG LSSPVTKSFN RGEC | 214 |

Some aspects and embodiments of the disclosure are described below in more detail with reference to the following examples, which are illustrative only and non-limiting.

### EXAMPLES

### Preparation of Drug Linkers

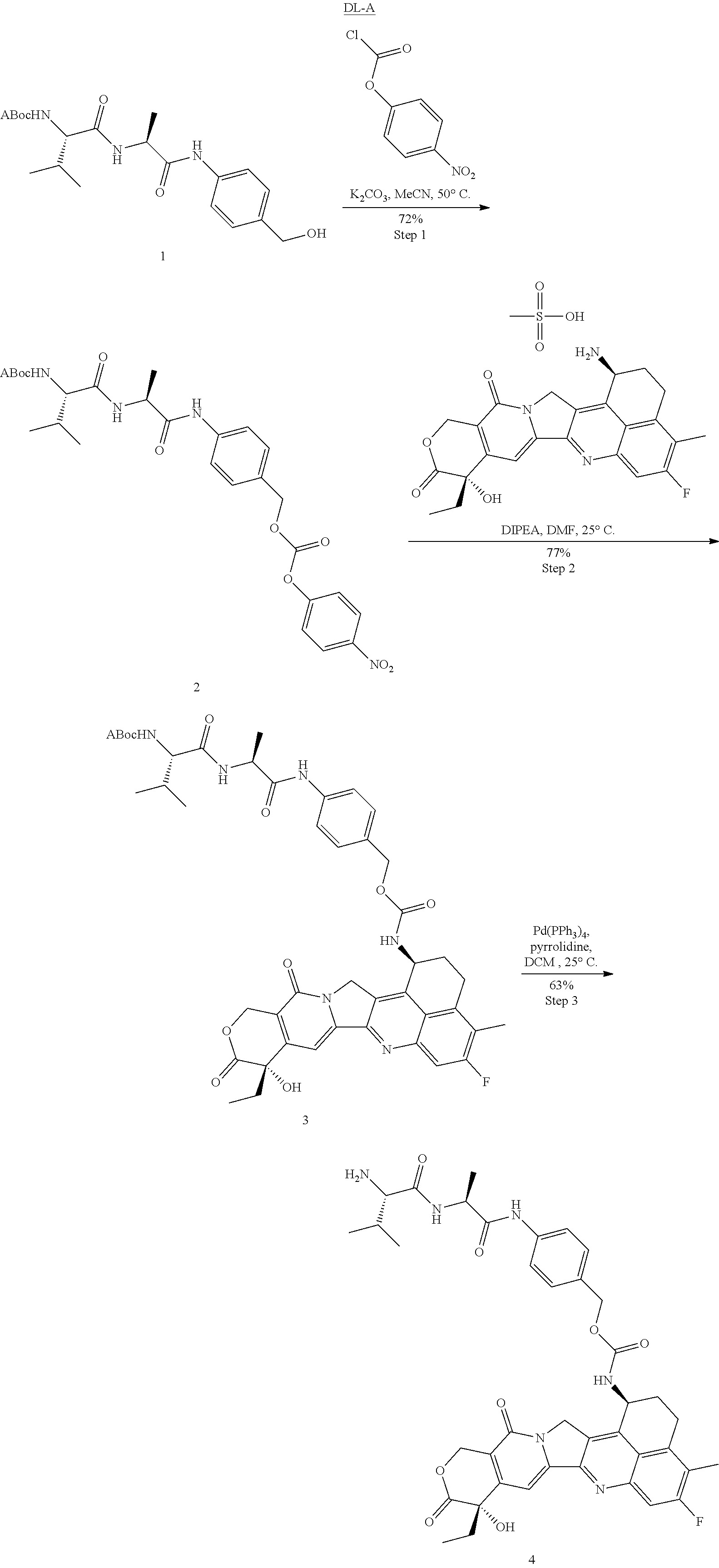

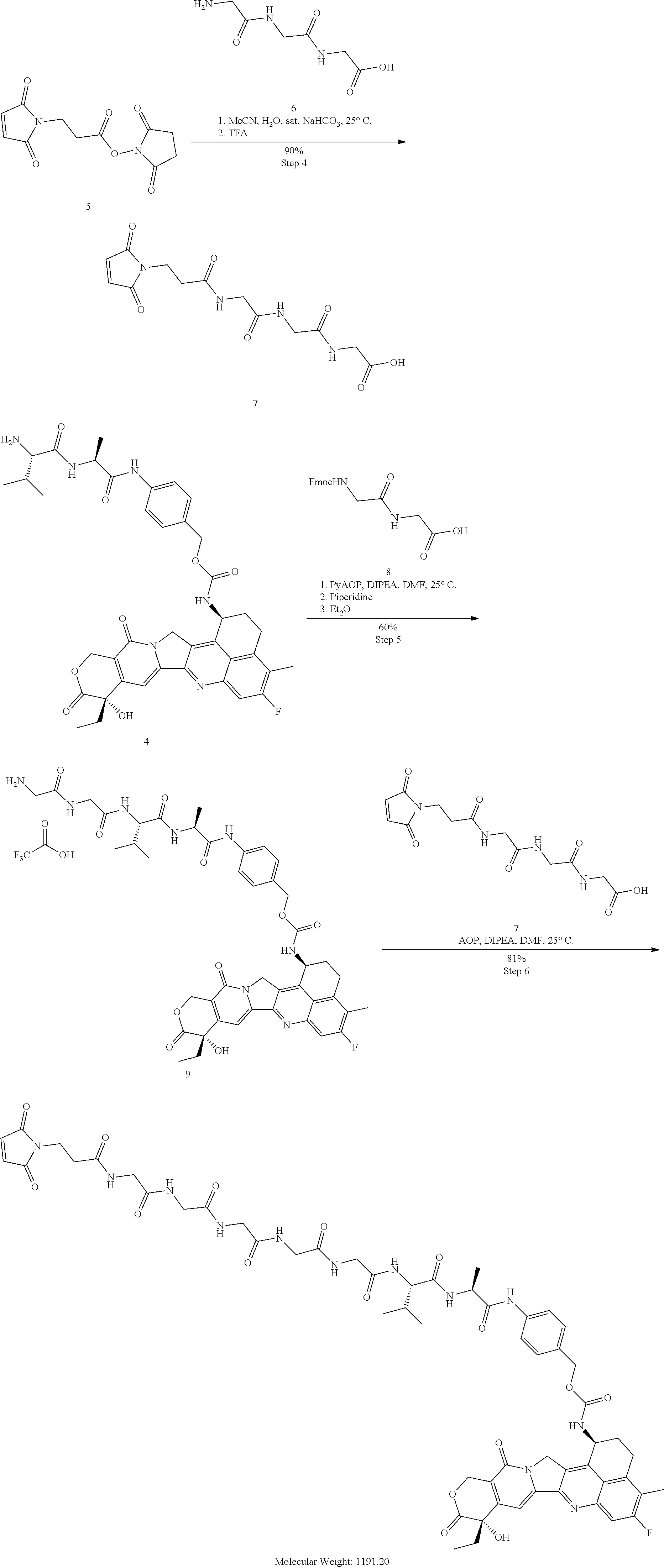

### Step 1:

Alloc-Val-Ala-PABA (1) (29 g, 1 eq) was dissolved in MeCN (10 V). K₂CO₃ (2 eq) was added. The reaction mixture was heated to 50° C. A solution of 4-nitrophenyl carbonochloridate in MeCN (2 eq in 5 V) was added. The reaction mixture was stirred at 50° C. for 6 h. On completion, the reaction mixture was filtered. The filtrate was concentrated in vacuo to give the crude residue. The latter was purified by flash column chromatography (silica) eluting with EtOAc/DCM (0→20%) to afford compound 2 (30 g, 72%).

### Step 2:

Exatecan mesylate (24 g, 1 eq) was dissolved in DMF (10 V). DIPEA (3 eq) was added. Compound 2 (1.25 eq) was added and the reaction mixture was stirred at 25° C. for 16 h.

On completion, water (30 V) was added dropwise to the reaction mixture. The latter was stirred at 25° C. for 30 min. The reaction mixture was filtered and the cake was washed with water (5 V), collected and thoroughly dried to afford compound 3 (29 g, 77%).

### Step 3:

Compound 3 (30 g, 1 eq) was dissolved in DCM (10 V). Pd (PPh₃)₄ (0.02 eq) and pyrrolidine (1.5 eq) were added. The reaction mixture was stirred at 25° C. for 2 h. On completion, the reaction mixture was concentrated in vacuo to give the crude residue. The latter was purified by flash column chromatography (silica) eluting with MeOH/DCM (0→8%) to afford compound 4 (17 g, 63%).

### Step 4:

To a solution of compound 5 (5.32 g, 20 mmol) and compound 6 (3.8 g, 20 mmol) in acetonitrile/water (1/2, v/v, 100 mL) was added sat. NaHCO₃ solution (40 mL).

The reaction was stirred at room temperature for 2 h. Then TFA (2.8 mL) was added and the stirring continued for an additional 10 min. The mixture was purified directly by preparative RP-HPLC to afford compound 7 as a white solid (6.1 g, 90%).

### Step 5:

To a solution of compound 4 (3.08 g, 4.08 mmol) and compound 8 (1.44 g, 4.08 mmol) in anhydrous DMF (50 mL) was added PyAOP (2.13 g, 4.08 mmol) and DIPEA

(2.1 mL, 12.08 mmol). The reaction was stirred at room temperature for 10 min. Then piperidine (2.0 mL) was added and the stirring continued for an additional 10 min.

The mixture was added to diethyl ether (500 mL) while stirring. The suspension was centrifugated and the precipitate was collected by decantation. Then the precipitate was dissolved in 40 mL of DMF and purified by preparative RP-HPLC to give compound 9 as a yellow solid, TFA salt (2.4 g, 60%).

### Step 6:

To a solution of compound 9 (TFA salt, 814 mg, 0.83 mmol) and compound 7 (282 mg, 0.83 mmol) in anhydrous DMF (20 mL) was added AOP (367 mg, 0.83 mmol) and DIPEA (0.57 mL, 3.28 mmol). The reaction was stirred at room temperature for 10 min. Then the mixture was purified by preparative RP-HPLC to afford compound DL-A as a yellow solid (510 mg, 81%).

### Preparative RP-HPLC Method:

Column: Phenomenex Gemini NX 5 m, C18, 110Å, 150×50 mm
Instrument: Shimadzu LC with CTC IFC
Mobile phase: A) water (0.1% TFA), B) acetonitrile
Gradient: 10-65% B over 20 min, flow 50 mL/min

### Analytical HPLC:

Column: Phenomenex Gemini NX 5 m, C18, 110Å, 150×4.6 mm
Instrument: Shimadzu LC-2030
Mobile phase: A) water (0.05% TFA), B) acetonitrile (0.05% TFA)
Gradient: 20-70% B over 15 min, flow 1.0 mL/min

All the desired HPLC fractions were pooled and lyophilised on a Virtis Freezemobile 35EL.

### Synthesis of ADCs

Different ADCs were constructed linked to antibody 2A10, which is an anti-PSMA antibody. The sequences of the 2A10 heavy and light chains are shown in SEQ ID NOs: 9 and 10, respectively.

ADC-1 has Exatecan as the warhead with a maleimide-Gly5-Val-Ala-PABA linker (DL-A) conjugated to antibody 2A10 via hinge region cysteine resides essentially as described in Zammarchi et al., 2016. Mol Cancer Ther 15:2709. In brief, antibody was buffer exchanged into a histidine buffer at pH 6 using tangential flow filtration, pH was adjusted to 7.5 using a TRIS/EDTA pH 8.5 buffer, and the solution was reduced with Tris (2-carboxyethyl) phosphine reductant. Dimethylacetamide and drug-linker (threefold excess relative to antibody) were added to the solution. The conjugation reaction was incubated, then quenched with threefold molar excess of N-acetyl cysteine and incubated again. The pH was then decreased to 6.0 using histidine hydrochloride solution and the generated ADC1was purified by tangential flow filtration, filtered, and stored at −70° C. Final yield was estimated by ultraviolet-visible spectrophotometry based on starting antibody. Synthesis of the drug linker DL-A is described above.

### Aggregation Studies

2 mg of a test monoclonal antibody at 8.87 mg/ml was pH adjusted with 5% 0.5 M Tris, 0.025 M EDTA, pH 8.5 and reduced with 2.3 mol. eq. TCEP and then conjugated with 6mol. eq. drug linker. After buffer exchange into 30 mM Histidine, 175 mM Sucrose pH 6.0/PBS, the drug to antibody ratio (DAR) was by HIC/LC-MS and % Monomer by size exclusion chromatography (SEC). Percentage monomer values of 95% or greater are assessed as low aggregation.

### Example 1—Aggregation Studies with Exatecan Linkers

Previous work that we have conducted using PEG-based linkers and an Exatecan warhead showed unfavourable aggregation properties. Therefore we sought to identify an alternative linker design. A new linker was designed using multiple glycine residues instead of PEG and this led to an improvement in levels of aggregation (data not shown). This glycine linker concept was then tested in new constructs based on a maleimide conjugation group and a Val-Ala-PABC cleavable portion linked to Exatecan.

A maleimide-Gly3-Val-Ala-PABC-Exatecan construct was tested against a maleimide-Gly5-Val-Ala-PABC-Exatecan construct (DL-A, the synthesis of which is described above-the Gly3 variant differs only with respect to the number of Gly residues). LogP values (a measure of hydrophobicity) were estimated using CDD Vault software (Collaborative Drug Discovery Inc.) based on the method described in Gedeck et al., 2017, J. Chem. Inf. Model. 57, 8, 1847-1858. Model training information using publicly available datasets is available from CDD. The results are shown in Table 1.

A correlation study was also carried out using Reverse-Phase HPLC (RP-HPLC). The two constructs were injected in RP-HPLC and retention times (Rt) correlated with hydrophobicity—i.e., high Rt was associated to hydrophobic constructs whereas low Rt to hydrophilic ones. The results are shown in Table 1.

Aggregation levels were assessed during the conjugation to a test antibody. Conjugation was performed as described above for ADC-1.

During conjugation, we are ideally targeting a monomer content or percentage monomer of 95% or above. Low percentage monomer values usually translate to a higher degree of aggregation. One could therefore agree that percentage monomer values of 95% or higher mean very low levels of aggregation or none, whereas values below this threshold often translate to higher levels of aggregation.

### Results

TABLE 1

| | | Rt | Aggregation |
|---|---|---|---|
| Construct ID | cLogP | (min) | (% monomer) |
| Mal-Gly5-VA-PABC-Exatecan | −0.81 | 9.76 | None |
| (DL-A-Gly5) | | | (98%) |
| Mal-Gly3-VA-PABC-Exatecan | 0.59 | 9.98 | Moderate |
| (DL-A-Gly3) | | | (87%) |

### Discussion

These results clearly show the hydrophilicity-enhancing properties in a drug linker construct of a novel pentaglycine spacer/linker. The incorporation of this Gly5 motif allowed constructs to exhibit increased hydrophilicity and lower aggregation levels for optimal conjugation processes. Gly5-based constructs showed no aggregation versus moderate aggregation for a Gly3-based version.

### Example 2—Assessment of Binding of Different Anti-PSMA Antibodies/Drug Conjugates

We screened alternative antibodies versus J591 using ELISA, cell-based PSMA binding studies (both with naked antibodies).

TABLE 2
Anti-PSMA antibodies tested

| | |
|---|---|
| J415 | BZL Biologics Inc.(Liu et al., |
| | 1997, Cancer Res. 57: 3629-3634) |
| MDX 2A10 | U.S. Pat. No. 7,875,278 |
| PMF-A10 | WO2010/037836 |
| Zona | WO2014/198223 |
| D2B | WO2009/130575 |

### ELISA Data

Human PSMA was coated on a 96 well plate at 3 μg/ml for at least two hours at room temperature. The plate was washed with wash buffer (0.05% Tween-20 in PBS), then blocked with blocking buffer (3% BSA in PBS) washed again using wash buffer. After which antibodies were titrated from 66.6 nM to 0.013 nM and added to the plate and incubated at room temperature for one hour, after which the plate was washed and anti-human-IgG-HRP added, the plate was incubated for a further 1 hour at room temperature, before being washed and TMB detection reagent added, the reaction was stopped by the addition of 1M HCl to the wells, after which the plate was read at 450 nm using the SpectraMax plate reader.

TABLE 3
Results

| PSMA Antibody | EC₅₀ (nM) |
|---|---|
| J415 | 24.84 |
| Mdx 2A10 | 0.1145 |
| PMF-A10 | 0.1615 |
| D2B | 0.173 |
| J591 | 0.2708 |
| ZONA | 0.3106 |

These results show that apart from the J415 antibody, all the tested antibodies bound with sub-nM EC₅₀with 2A10 have the lowest (best) value.

### Example 3-In Vivo Efficacy of Anti-PSMA Antibody Conjugated to an Exatecan Payload in the Androgen-Sensitive Metastatic Prostate Cancer LNCaP Model

Male immunodeficient mice (Athymic Nude-Foxn1nu, Crown Bio) were seven to nine weeks old with a minimum body weight (BW) of 15 g on Day 1 of the study. Each mouse was injected subcutaneously (s.c.) in the right flank with 5×10⁶ cells in 50% matrigel. Tumors were measured in two dimensions using calipers, and the volume was calculated using the formula:

$$Tumor Volume ( {mm}^{3} ) = {w}^{2} \times l / 2$$

where w=width and l=length, in mm, of the tumor. Tumor weight may be estimated with the assumption that 1 mg is equivalent to 1 mm³ of tumor volume.

Nineteen days later, designated as Day 1 of the study, mice were sorted into treatment groups (n=10 per group) with individual tumor volumes typically ranging from 150 to 200 mm³. On Day 1 of the study, drugs were administered intravenously (i.v.) in a single injection (qd×1) via tail vein injection. The dosing volume was 0.2 mL per 20 grams of body weight (10 mL/kg) and was scaled to the body weight of each individual animal. Tumors were measured using calipers twice per week, and each animal was euthanized when its tumor reached the endpoint (mean diameter 15 mm) or at the end of the study (Day 35), whichever came first.

B12 is a control antibody that binds to HIV gp120.

TABLE 4
Results

| | | | % |
|---|---|---|---|
| Response summary LNCaP | Day 1 | Day 35 | change |
| Vehicle | 156 | 1212 | 677 |
| 2A10-DL-A (10 mg/kg) (Exatecan) | 148 | 174 | 18 |
| 2A10-DL-A (5 mg/kg) (Exatecan) | 149 | 249 | 67 |
| B12-DL-A (10 mg/kg) (Exatecan) | 160 | 1297 | 711 |

### Discussion

2A10-DL-A shows high anti-tumour activity in the androgen-sensitive metastatic prostate cancer LNCaP model. The treatment was well tolerated.

A number of publications are cited above to more fully describe and disclose the disclosures and the state of the art to which inventions herein may pertain. The entirety of each of the references mentioned in this disclosure are hereby incorporated by reference.

1. An antibody drug conjugate of formula (I):
Ab-L-Dp (I)
wherein:
Ab is an antibody that binds to PSMA, which antibody comprises (i) an immunoglobulin heavy chain variable region having a CDR1 region with the amino acid sequence shown in SEQ ID NO: 3, a CDR2 region with the amino acid sequence shown in SEQ ID NO: 4, and a CDR3 region with the amino acid sequence shown in SEQ ID NO: 5; and (ii) an immunoglobulin light chain variable region having a CDR1 region with the amino acid sequence shown in SEQ ID NO: 6, a CDR2 region with the amino acid sequence shown in SEQ ID NO: 7, and a CDR3 region with the amino acid sequence shown in SEQ ID NO: 8;
L-Dp is a drug linker conjugated to the Ab, wherein L consists of-G^{LL}-X-Glyn-A-, where G^{LL}is for connection of the linker to a cysteine residue on the antibody, X is —(CH₂)₁₋₅—C(O)—, Glyₙ is a polyglycine moiety wherein n is from 5 to 8, and A is a cathepsin-cleavable moiety linked to a self-immolative moiety; and D is an Exatecan, wherein the drug linker can be cleaved under cellular conditions to release an Exatecan with the following formula (II): 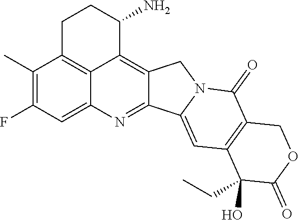 [SMILES: CC[C@@]1(O)C(=O)OCc2c1cc1n(c2=O)Cc2c-1nc1cc(F)c(C)c3c1c2[C@@H](N)CC3]
and wherein p is the number of drug units per antibody and is from 1 to 6.

2. A conjugate according to claim 1 wherein the antibody has a VH domain as shown in SEQ ID NO: 1 and a VL domain as shown in SEQ ID NO: 2.

3. A conjugate according to claim 1 wherein the cathepsin cleavable sequence is Val-Ala or Val-Cit.

4. A conjugate according claim 1 wherein the self-immolative moiety is para-aminobenzylcarbamate.

5. A conjugate according to claim 1 wherein G^{LL} is:

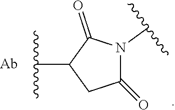 [SMILES: *C1CC(=O)N(*)C1=O]

6. A conjugate according to claim 1 where L-Dp is:

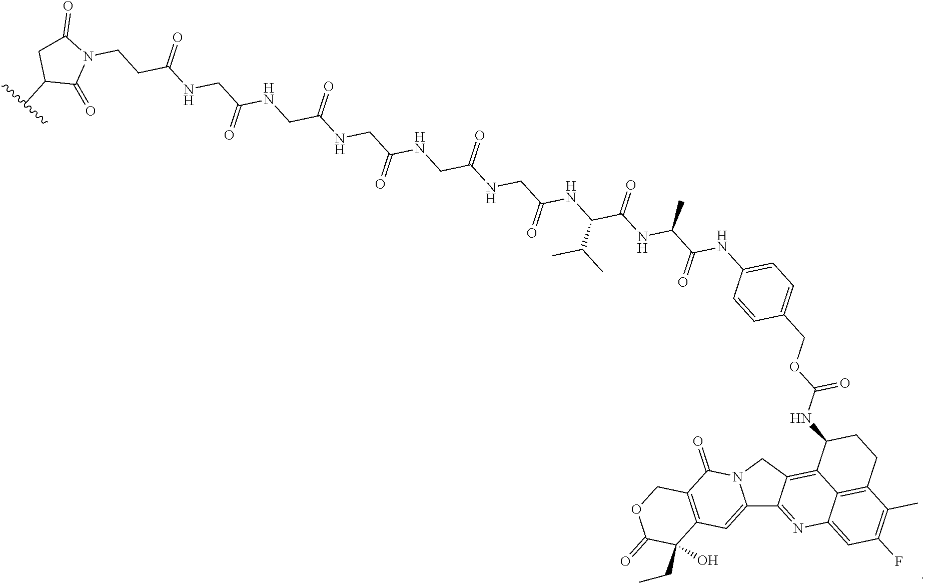 [SMILES: *C1CC(=O)N(CCC(=O)NCC(=O)NCC(=O)NCC(=O)NCC(=O)NCC(=O)N[C@H](C(=O)N[C@@H](C)C(=O)Nc2ccc(COC(=O)N[C@H]3CCc4c(C)c(F)cc5nc6c(c3c45)Cn3c-6cc4c(c3=O)COC(=O)[C@]4(O)CC)cc2)C(C)C)C1=O]

7. A conjugate according to claim 6 wherein the antibody has a VH domain as shown in SEQ ID NO: 1 and a VL domain as shown in SEQ ID NO: 2.

8. A composition comprising a mixture of antibody drug conjugates according to claim 1 wherein the average drug to antibody ratio is from 3 to 6.

9. A method of treating an individual suffering from a proliferative disease characterized by over-expression of PSMA which method comprises administering to the patient a conjugate according to claim 1.

10. A method of treating an individual suffering from prostate cancer, which method comprises administering to the patient a conjugate according to claim 1.


SEQUENCE LISTING (18 sequences)

SEQ ID NO: 1 - protein, 119 aa, synthetic construct
EVQLVQSGAEVKKPGESLKISCKGSGYSFTSNWIGWVRQMPGKGLEWMGIIYPGDSDTRYSPSFQGQVTISADKSISTAYLQWSSLKASDTAMYYCARQTGFLWSSDLWGRGTLVTVSS

SEQ ID NO: 2 - protein, 107 aa, synthetic construct
AIQLTQSPSSLSASVGDRVTITCRASQDISSALAWYQQKPGKAPKLLIYDASSLESGVPSRFSGYGSGTDFTLTINSLQPEDFATYYCQQFNSYPLTFGGGTKVEIK

SEQ ID NO: 3 - protein, 5 aa, synthetic construct
SNWIG

SEQ ID NO: 4 - protein, 17 aa, synthetic construct
IIYPGDSDTRYSPSFQG

SEQ ID NO: 5 - protein, 10 aa, synthetic construct
QTGFLWSSDL

SEQ ID NO: 6 - protein, 11 aa, synthetic construct
RASQDISSALA

SEQ ID NO: 7 - protein, 7 aa, synthetic construct
DASSLES

SEQ ID NO: 8 - protein, 9 aa, synthetic construct
QQFNSYPLT

SEQ ID NO: 9 - protein, 448 aa, synthetic construct
EVQLVQSGAEVKKPGESLKISCKGSGYSFTSNWIGWVRQMPGKGLEWMGIIYPGDSDTRYSPSFQGQVTISADKSISTAYLQWSSLKASDTAMYYCARQTGFLWSSDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
AIQLTQSPSSLSASVGDRVTITCRASQDISSALAWYQQKPGKAPKLLIYDASSLESGVPSRFSGYGSGTDFTLTINSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 4 aa, synthetic construct
GGFG

SEQ ID NO: 12 - protein, 5 aa, synthetic construct
  REPEAT 5: note = Glycine at position 5 may be repeated 1 to 3 times to give a range of 5 to 8 glycine residues in total
GGGGG

SEQ ID NO: 13 - protein, 5 aa, synthetic construct
GGGGG

SEQ ID NO: 14 - protein, 6 aa, synthetic construct
GGGGGG

SEQ ID NO: 15 - protein, 7 aa, synthetic construct
GGGGGGG

SEQ ID NO: 16 - protein, 8 aa, synthetic construct
GGGGGGGG

SEQ ID NO: 17 - protein, 7 aa, synthetic construct
GGGGGVA

SEQ ID NO: 18 - protein, 5 aa, synthetic construct
GGGVA